(19) United States
(12) Patent Application Publication
(10) Pub. No.: US 2023/0118421 A1
(43) Pub. Date: Apr. 20, 2023

(54) MOTHER AND CHILD HEALTH MANAGEMENT SYSTEM AND INFORMATION PROVISION METHOD

(71) Applicants: NATIONAL UNIVERSITY CORPORATION HOKKAIDO UNIVERSITY, Sapporo-shi, Hokkaido (JP); MORINAGA MILK INDUSTRY CO., LTD., Tokyo (JP); Hitachi, Ltd., Tokyo (JP)

(72) Inventors: Takahiro NAKAMURA, Tokyo (JP); Takashi TAKEMOTO, Tokyo (JP); Masanori YOSHINO, Tokyo (JP); Tokiyoshi AYABE, Sapporo-shi, Hokkaido (JP); Takashi KIMURA, Sapporo-shi, Hokkaido (JP); Hirohisa IZUMI, Kanagawa (JP); Fuka TABATA, Kanagawa (JP)

(21) Appl. No.: 17/910,885

(22) PCT Filed: Mar. 12, 2021
(86) PCT No.: PCT/JP2021/010011

§ 371 Date: Sep. 12, 2022

(30) Foreign Application Priority Data
Jun. 19, 2020 (JP) 2020-106028

(51) Int. Cl. G16H 50/30; G16H 10/60

(52) U.S. Cl. CPC G16H 50/30; G16H 10/60; G06Q 50/10

(57) ABSTRACT

A mother and child health management system is connected to a plurality of terminals and includes: a reception unit configured to receive, as analysis information, information from each of the plurality of terminals and biological information from a medical care institution; a classification unit configured to identify, based on an analysis result of the analysis information, state values indicating a state of a mother, a state of a child, and a state between the mother and the child; an access authority management section configured to identify, based on each of the state values, a community to which a family including a user of the each of the plurality of terminals belongs, and configured to provide the terminal with an access authority to receive a display of a solution related to the community; and a display unit configured to display the solution related to the community on the terminal.

### CROSS-REFERENCE TO RELATED APPLICATIONS

The present invention claims the benefit of priority to Japanese Patent Application No. 2020-106028 (filed on Jun. 19, 2020) with the Japanese Patent Office, the entire contents of which are incorporated herein by reference in its entirety.

### TECHNICAL FIELD

The present invention relates to a system configured to analyze and manage health of a mother or child.

### BACKGROUND ART

A cohort study has been performed to investigate lifestyles and health states of a mother and a child from pregnancy until after childbirth, for purpose of supporting a healthy development of the child or the like. A health management system, used for the cohort study to investigate a cause and effect of a disease or others, is configured to conduct investigation and analysis, and concurrently configured to display advice to a user for quality of life improvement (see, for example, PTL 1).

PTL 1 discloses “a health information management system first registers registration information related to a monitoring target in a monitoring target information storage unit 151. Next, the health information management system sets up a health level based on the registration information, and stores biological information, which has been measured based on the health level and transmitted from a monitoring unit, in a biological information storage unit 153. Based on the biological information stored in the biological information storage unit 153, the health information management system executes processing to determine whether or not the monitoring target is in a normal state. Then, in accordance with the determination, the health information management system executes processing to display the advice to the monitoring target.”

### CITATION LIST

### Patent Literature

PTL 1: JP 2003-310560 A

### SUMMARY OF INVENTION

### Technical Problem

Particularly, at an early stage of the cohort study, it is difficult to generate highly reliable advice due to insufficient clinical trials. Thus, the user does not fully enjoy benefits of participating in the cohort study, which hardly motivates the user to further use the health management system of the cohort study.

An object of the present invention is to establish a mother and child health management system as a system to analyze and manage health of a mother or child, the system where a user enjoys benefits and feels encouraged to further use the system.

### Solution to Problem

In this application, the present invention discloses a representative aspect as follows. Provided is a mother and child health management system provided with at least one computer and connected to a plurality of terminals, through which the mother and child health management system is used,

the mother and child health management system includes:
a storage unit configured to store community information to manage a community defined by a combination of a state value indicating a state of a mother, a state value indicating a state of a child, and a state value indicating a state between the mother and the child, and solution information to manage a solution for a concern shared in the community;
a reception unit configured to receive, as analysis information, information inputted as a text from each of the plurality of terminals, the information related to at least any one of the state of the mother and the state of the child, the mother and the child of a family including a user of the each of the plurality of terminals as a member, and biological information from a medical care institution, the biological information related to at least any one of the mother and the child;
a classification unit configured to identify, based on an analysis result of the analysis information, the state value of the mother, the state value of the child, and the state value between the mother and the child, and configured to output each of the state values as state information;
an access authority management section configured to identify a community, to which the family of the user of the terminal belongs, by referring to the community information based on the state information, and configured to provide the terminal with an access authority to receive a display of the solution for the concern shared in the community; and
a solution display section configured to display on the terminal the solution for the concern shared in the community.

In the mother and child health management system, on receipt, from the terminal, of a vote to evaluate the solution for the concern shared in the community to which the family of the user of the terminal belongs, the access authority management section removes from the terminal the access authority to the community to which the family belongs.

### Advantageous Effects of Invention

The present invention provides a mother and child health management system where a user enjoys benefits and thus feels encouraged to further use the system. Problems, configurations, and effects in addition to those described above will be clarified below in descriptions of embodiment.

### BRIEF DESCRIPTION OF DRAWINGS

FIG. 1 is a diagram showing an example of a configuration of a mother and child health management system according to a first embodiment.

FIG. 2 is a diagram showing an example of a configuration of data stored in a community database (DB) according to the first embodiment.

FIG. 3 is a diagram showing an example of a configuration of data stored in a local health database (DB) according to the first embodiment.

FIG. 4 is a diagram showing an example of a configuration of data stored in a solution database (DB) according to the first embodiment.

FIG. 5 is a diagram showing an example of a screen displayed on a mobile terminal according to the first embodiment.

FIG. 6 is a diagram showing an example of a screen displayed in a local government system according to the first embodiment.

FIG. 7 is a diagram showing an example of a registration screen displayed by the mother and child health management system according to the first embodiment.

### DESCRIPTION OF EMBODIMENTS

An embodiment of the present invention will be described below with reference to the drawings. Note that, the present invention is not limited to the embodiment below. Thus, it is easily understood for those skilled in the art that any change, addition, or deletion of a configuration of each unit appropriately made within the spirit of the present invention will naturally fall within the scope of claims of the present invention. In descriptions below, same names and reference signs represent identical or equivalent components or functions, and a detailed description thereof will be omitted as appropriate. In this specification, a description, such as “first”, “second”, or “third” is provided for convenience of identifying each component, and does not necessarily restrict the quantity or order. A position, size, shape, range, or the like of each component illustrated in the drawings may not necessarily represent an actual position, size, shape, range, or the like, in order to facilitate understanding of the present invention. Accordingly, the present invention is not necessarily limited to the position, size, shape, range, or the like disclosed in the drawings.

### First Embodiment

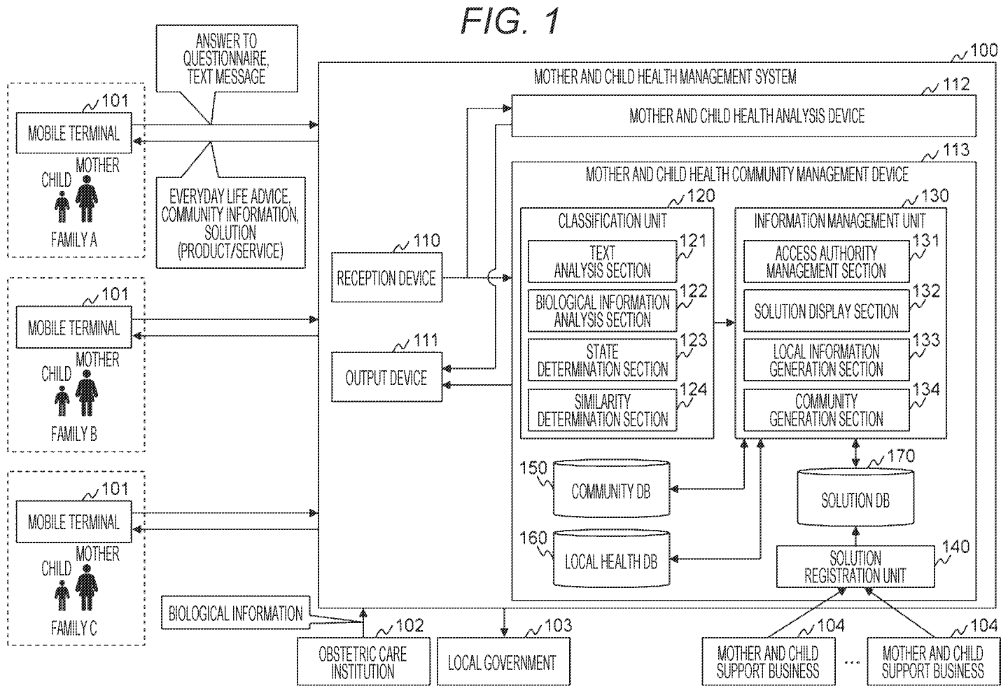

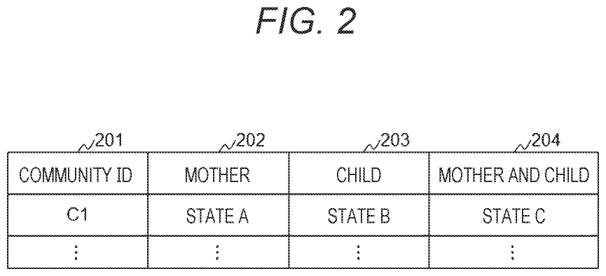

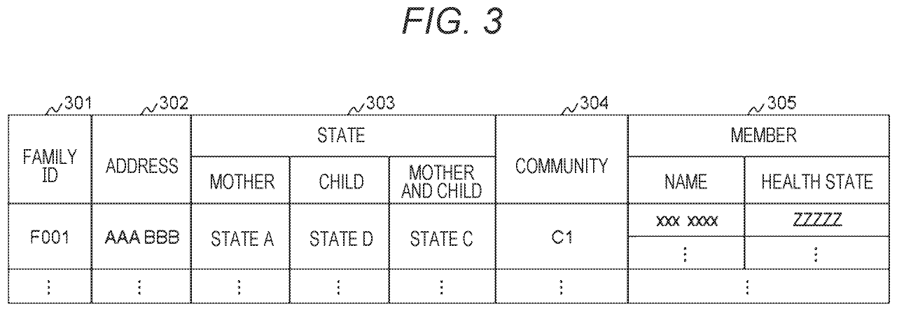

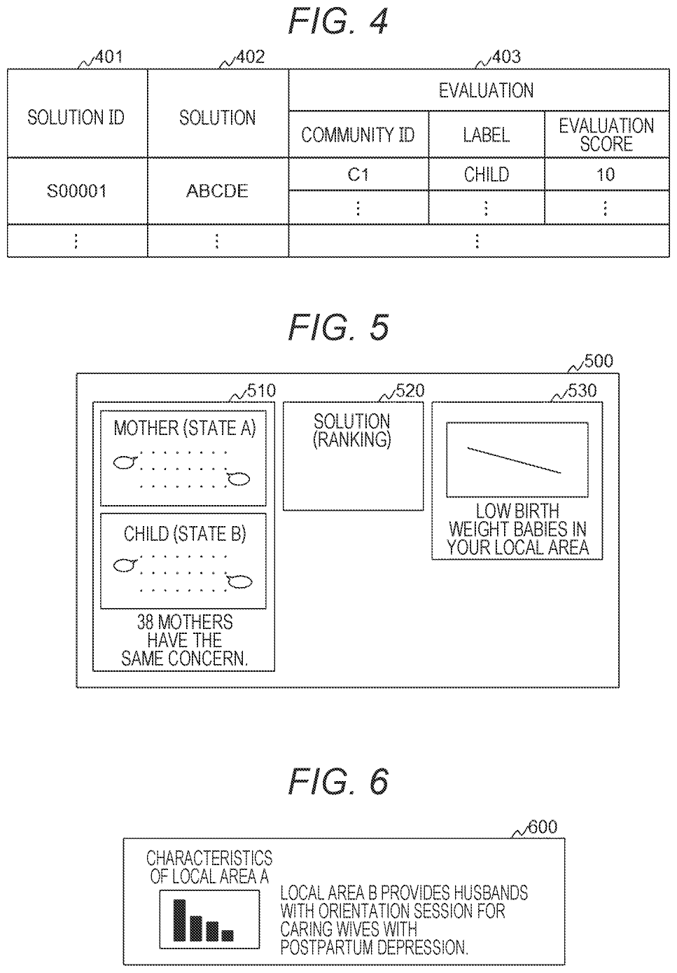

FIG. 1 is a diagram showing an example of a configuration of a mother and child health management system according to a first embodiment. FIG. 2 is a diagram showing an example of a configuration of data stored in a community DB 150 according to the first embodiment. FIG. 3 is a diagram showing an example of a configuration of data stored in a local health DB 160 according to the first embodiment. FIG. 4 is a diagram showing an example of a configuration of data stored in a solution DB 170 according to the first embodiment.

A mother and child health management system 100 is connected to a plurality of mobile terminals 101, each of which a family including a mother and a child as its member uses. In the descriptions below, when the member of the family, using a corresponding one of the plurality of mobile terminals 101, is not particularly specified, the member will be referred to as a user.

The mother and child health management system 100 and the mobile terminal 101 are connected via a network such as a wide area network (WAN) or a local area network (LAN). The mother and child health management system 100 and the mobile terminal 101 may be connected via either wired or wireless link. Alternatively, the mother and child health management system 100 and the mobile terminal 101 may be directly connected to each other.

The mother and child health management system 100 is connected to a local government system 103 and an obstetric care institution system 102 that the mother and the child use. Further, the mother and child health management system 100 is connected to a mother and child support business system 104 configured to provide at least any one of a service and a product to support the mother and the child. The mother and child health management system 100 may be connected to the obstetric care institution system 102, the local government system 103, and the mother and child support business system 104 via the network, or alternatively, may be directly connected thereto.

The mobile terminal 101 is a terminal such as a smartphone. The user uses the mobile terminal 101 to input an answer to a questionnaire and a text. For example, the mother as the user inputs a concern about her child care or others as the text. Further, the user uses the mobile terminal 101 to browse advice related to everyday life, information related to a community, a solution related to the community, or others transmitted from the mother and child health management system 100.

The user may use a personal computer, a television, or others instead of the mobile terminal 101.

The obstetric care institution system 102, including at least one computer, is configured to transmit biological information related to a health state of the mother and a health state of the child in response to a request from the mother and child health management system 100. Note that, the biological information is transmitted on an assumption that a privacy contract has been previously made between the user (e.g., the mother) and the mother and child health management system 100. The biological information includes, for example, data related to breast milk, feces, and blood.

The local government system 103, including at least one computer, is configured to acquire local information or others from the mother and child health management system 100.

The mother and child support business system 104, including at least one computer, is configured to register the service or the product in the mother and child health management system 100.

The mother and child health management system 100 includes a reception device 110, an output device 111, a mother and child health analysis device 112, and a mother and child health community management device 113, and is configured to analyze and manage health of the mother or child.

The reception device 110 is a computer including a central processing unit (CPU) (not illustrated), a memory, a network interface, or others. The reception device 110 may have a storage unit such as a hard disk drive (HDD) or a solid state drive (SSD), may have an input unit such as a keyboard, a mouse, or a touch panel, or may have an output unit such as a display.

The CPU executes a program stored in the memory. The CPU executes processing in accordance with the program to operate as a functional unit (module) that carries out a specific function. The memory stores the program that the CPU executes as well as data that the program uses. The memory is also used as a work area that the program temporarily uses. The network interface is an interface to be connected to an external device via the network.

The reception device 110 is configured to receive information and a request from the external device or an external system. The reception device 110 is also configured to transmit the information and the request received to the mother and child health analysis device 112 and to the mother and child health community management device 113.

For example, the reception device 110 receives from the mobile terminal 101 the answer to the questionnaire and the text, and transmits the answer to the questionnaire and the text to the mother and child health analysis device 112 and the mother and child health community management device 113. The reception device 110 receives the biological information from the obstetric care institution system 102, and transmits the biological information to the mother and child health analysis device 112 and the mother and child health community management device 113. The reception device 110 receives an information acquisition request from the local government system 103, and transmits the information acquisition request to the mother and child health community management device 113. The reception device 110 receives a solution registration request from the mother and child support business system 104, and transmits the solution registration request to the mother and child health community management device 113.

The output device 111 is a computer including the CPU (not illustrated), the memory, the network interface, or others. The output device 111 may have the storage unit, the input unit, or the output unit.

The output device 111 receives various information from the mother and child health analysis device 112 and the mother and child health community management device 113, and outputs the various information to the external device or the external system.

For example, the output device 111 receives the questionnaire and the advice related to everyday life from the mother and child health analysis device 112, and transmits the questionnaire and the advice related to everyday life to the mobile terminal 101. The output device 111 receives the information and the solution related to the community from the mother and child health community management device 113, and transmits the information and the solution related to the community to the mobile terminal 101. The output device 111 receives the local information from the mother and child health community management device 113, and transmits the local information to the local government system 103.

The mother and child health analysis device 112 is a computer including the CPU (not illustrated), the memory, the network interface, or others.

The mother and child health analysis device 112 may have the storage unit, the input unit, or the output unit. The mother and child health analysis device 112 is configured to generate the questionnaire to grasp the health states of the mother and the child. The mother and child health analysis device 112 is also configured to analyze the health states of the mother and the child based on the answer to the questionnaire, and configured to generate the advice related to everyday life based on an analysis result of the health states of the mother and the child.

The mother and child health community management device 113 is a computer including the CPU (not illustrated), the memory, the network interface, or others. The mother and child health community management device 113 may have the storage unit, the input unit, or the output unit.

The mother and child health community management device 113 is configured to manage each of the communities and determine a community to which the family belongs.

Here, the community corresponds to a group of families including users having certain characteristics. The community is, for example, a community of families including mothers having the same concern, such as a community of families including children suffering from a specific disease, a community of families including mothers suffering from postpartum depression, or a community of families including mothers having difficulties with babies' cries at night.

The mother and child health community management device 113 has a functional configuration as will be described below.

The mother and child health community management device 113 holds the community DB 150, the local health DB 160, and the solution DB 170 as information.

The community DB 150 stores information to define the community. Specifically, the community DB 150 stores a plurality of records as shown in FIG. 2. Each of the plurality of records is stored with regard to the corresponding community. Each of the plurality of records includes a community ID 201, a mother 202, a child 203, and a mother and child 204.

The community ID 201 is a field for storing identification information of the community. The mother 202 is a field for storing a state value indicating the health state of the mother. The child 203 is a field for storing a state value indicating the health state of the child. The mother and child 204 is a field for storing a state value indicating a relationship between the mother and the child, such as a degree of intimacy, the relationship not included in the health states of the mother and the child.

As has been described above, each community is defined by a combination of the state values indicating the state of the mother, the state of the child, and the state between the mother and the child.

The field of the mother may be replaced with a field of a father. Alternatively, the record includes both the field of the mother and the field of the father.

The local health DB 160 stores information related to the health states of the members of the family. Specifically, the local health DB 160 stores a plurality of records as shown in FIG. 3. Each of the plurality of records is stored with regard to the corresponding family. Each of the plurality of records includes a family ID 301, an address 302, a state 303, a community 304, and a member 305.

The family ID 301 is a field for storing identification information of the family. The address 302 is a field for storing an address corresponding to a residential location of the family. The state 303 is a field for storing each of the state values of the mother, the child, and between the mother and the child. The community 304 is a field for storing the identification information of the community to which the family belongs. The community 304 stores as many community IDs as the number of the communities to which the family belongs. The member 305 is a field for storing information related to the members of the family. The member 305 includes as many rows as the number of the members of the family, each of the rows including a name and a health state of the corresponding member. Note that, it is naturally to be understood that the information above needs to be handled with care, such as protection of security, on an assumption that the user has agreed in terms of personal information protection. Here, instead of the address, the record may include information indicating only a local area where the family resides.

The mother and child health community management device 113 may hold a database configured to manage a history of the health states.

The solution DB 170 stores information related to a solution for a concern shared in the community. Specifically, the solution DB 170 stores a plurality of records as shown in FIG. 4. Each of the plurality of records is stored with regard to the corresponding solution. Each of the plurality of records includes a solution ID 401, a solution 402, and an evaluation 403.

The solution ID 401 is a field for storing identification information of the solution. The solution 402 is a field for storing a specific content of the solution. The evaluation 403 is a field for storing the evaluation for the solution in each of the communities, and includes a plurality of rows, each including the community ID, a label, and an evaluation score.

Here, the label corresponds to information indicating the user to whom the solution is applied; the label is, for example, the “mother”, the “child”, the “mother and child”, or others. The record may not necessarily include the label. The evaluation score corresponds to a value indicating a quality of the solution for the community corresponding to the community ID.

The mother and child health community management device 113 includes a classification unit 120, an information management unit 130, and a solution registration unit 140 as functional units.

The classification unit 120, the information management unit 130, and the solution registration unit 140 are the functional units run by the CPU (not illustrated) that executes the programs stored in the memory (not illustrated). In the descriptions below, when it is explained that each of the classification unit 120, the information management unit 130, and the solution registration unit 140 carries out some processing, this explanation indicates that the CPU (not illustrated) executes the program for the corresponding functional unit.

The classification unit 120 is configured to identify each of the state values indicating the state of the mother, the state of the child, and the state between the mother and the child, based on the answer to the questionnaire and the text acquired from the user of the mobile terminal 101, and based on the biological information related to at least any one of the mother and the child included in the family to which the user belongs as a member. The classification unit 120 is also configured, based on each of the state values, to determine whether or not the family is similar to each of the communities managed in the community DB 150.

The classification unit 120 includes a text analysis section 121, a biological information analysis section 122, a state determination section 123, and a similarity determination section 124.

The text analysis section 121 executes text analysis processing based on the answer to the questionnaire and the text, so as to extract items (e.g., a character string) related to each of the states. For example, when the text inputted refers to the babies' cries at night, the text analysis section 121 extracts “babies' cries at night” as the item related to the state between the mother and the child. In this text analysis, the items may be extracted based on a typically-used natural language processing technique.

The biological information analysis section 122 executes biological information analysis processing based on the biological information, so as to calculate the state value related to each of the states. For example, when the biological information corresponds to the data related to the feces, the biological information analysis section 122 calculates a state of a gut microbiome (e.g., bacterial species diversity such as a diversity and β, diversity, and α-defensin concentration).

The state determination section 123 identifies the state value indicating each of the states (of the mother, of the child, and between the mother and the child) based on the result of each analysis processing.

The similarity determination section 124 calculates a degree of similarity of the family to each of the communities, based on the state value indicating each of the states, the state value used to define the community. For example, the similarity determination section 124 calculates, as the degree of similarity, a Euclidean distance in a feature space.

The information management unit 130 is configured to determine the community to which the family belongs and display the solution related to the community on the mobile terminal 101. The information management unit 130 is also configured to generate the local information and transmit the local information to the local government system 103.

The information management unit 130 includes an access authority management section 131, a solution display section 132, a local information generation section 133, and a community generation section 134.

The access authority management section 131 determines the community, to which the family of the user of the mobile terminal 101 belongs, and provides the mobile terminal 101 with an access authority to view, register, and evaluate the solution related to the community determined.

The solution display section 132 displays the solution on the mobile terminal 101 of the user included in the family that belongs to the community.

The local information generation section 133 executes statistical processing related to the community to generate the local information, and transmits the local information to the local government system 103.

The community generation section 134 executes grouping based on the state value indicating each of the states in each of a plurality of the families, so as to generate the corresponding community.

The solution registration unit 140 is configured to register the solution in the solution DB 170 in response to the solution registration request from the mobile terminal 101 or the mother and child support business system 104.

Next, each processing executed by the mother and child health community management device 113 will be described in detail.

(1) First, in order to determine the community to which the family belongs, the mother and child health community management device 113 executes the processing, as will be described below.

When receiving, from the reception device 110, the answer to the questionnaire and the text from the mobile terminal 101 of the user included in each of the families, the mother and child health community management device 113 starts the processing.

The classification unit 120 acquires the biological information from the obstetric care institution system 102. Specifically, the classification unit 120 transmits to the obstetric care institution system 102 the information acquisition request, along with the family ID that has been transmitted together with the answer to the questionnaire and the text.

The text analysis section 121 of the classification unit 120 executes the text analysis processing based on the answer to the questionnaire and the text. Additionally, in the classification unit 120, the biological information analysis section 122 executes the biological information analysis processing based on the answer to the questionnaire and the biological information. The text analysis processing and the biological information analysis processing are known techniques, and a detailed description thereof will thus be omitted as appropriate. Note that, the biological information may not be acquired, or alternatively, the answer to the questionnaire and the text may not be acquired.

The state determination section 123 of the classification unit 120 identifies the state value indicating each of the states (of the mother, of the child, and between the mother and the child) based on the result of each analysis processing. In some cases, any one of the states may not exist. In this case, the state that does not exist is described as 0 or NULL, or shown as a blank.

The similarity determination section 124 of the classification unit 120 calculates the degree of similarity of the family to the each of the communities that has been defined in the corresponding record of the community DB 150. The similarity determination section 124 outputs, to the information management unit 130, output information including the family ID and the degree of similarity of the family to each of the communities.

Based on the output information, the access authority management section 131 of the information management unit 130 determines the community to which the family belongs. For example, the access authority management section 131 determines any one of the communities, which shows the degree of similarity higher than a threshold value, as the community to which the family belongs. Here, two or more of the communities may be determined as the community. Alternatively, none of the communities may be determined as the community.

The access authority management section 131 of the information management unit 130 provides the mobile terminal 101 with the access authority to the community to which the family belongs. Specifically, the access authority management section 131 refers to the local health DB 160 to search for the record in correspondence to the family ID, and registers the community ID in the community 304 of the record searched. In this state, the access authority management section 131 of the information management unit 130 may notify the mobile terminal 101 of the community information. When none of the communities is determined as the community to which the family belongs, the access authority management section 131 registers an undefined community ID in the community 304 of the record searched.

As has been described above, when any one of the answer to the questionnaire, the text, and the biological information has been inputted, the community, to which the family (user) belongs, is automatically classified.

(2) Next, in order to display the solution on the mobile terminal 101, the mother and child health community management device 113 executes the processing as will be described below.

When receiving, from the mobile terminal 101, a solution display request, or when the community has been determined, the mother and child health community management device 113 starts the processing described below.

The solution display section 132 of the information management unit 130 refers to the local health DB 160 to identify the community to which the family including the user of the mobile terminal 101 belongs. Specifically, the solution display section 132 searches for the record of the family including the user of the mobile terminal 101 based on the name or the family ID of the user, and acquires the community ID stored in the community 304 of the corresponding record.

The solution display section 132 of the information management unit 130 uses the solution DB 170 to generate solution display information. Specifically, the solution display section 132 refers to the solution DB 170 to search for the record including a row where the corresponding community ID is stored in the community ID column of the evaluation 403. Based on the evaluation score for the community of the record searched, the solution display section 132 selects a predetermined number of the records, and sorts the predetermined number of records in descending order from highest to lowest of these evaluation scores. The solution display section 132 generates, as the solution display information, a list of the solutions stored in the records in the sorted order.

For example, the solution display section 132 selects the records where the evaluation scores are higher than a threshold value. Then, the solution display section 132 selects the predetermined number of records in descending order from highest to lowest of these evaluation scores.

The solution display section 132 of the information management unit 130 transmits the solution display information to the mobile terminal 101.

The solution display section 132 may generate the community information and transmit the community information to the mobile terminal 101. Specifically, the solution display section 132 refers to the local health DB 160 based on the community ID, and counts the number of the records, where the corresponding community ID is stored in community 304, as the number of the families that belong to the community. The solution display section 132 generates the community information that includes the state values in the state 303 of the records searched with reference to the local health DB 160 as well as the number of the families belonging to the community.

The mobile terminal 101 displays a screen based on the solution display information and the community information. FIG. 5 is a diagram showing an example of the screen displayed on the mobile terminal 101 according to the first embodiment.

A screen 500 includes a display column 510, a display column 520, and a display column 530. The display column 510 displays, based on the community information, the state of the mother, the state of the child, and the number of the families that belong to the community. The screen may have a configuration where the user of the mobile terminal 101 is allowed to freely input a chat message such that the users share the information in the community. In this case, the user of the mobile terminal 101 may belong to a plurality of the communities and thus, the chat message may be displayed in a plurality of ranges, such as the mother (state A) and the child (state B), as displayed in FIG. 5. The display column 520 displays the solutions in a ranking order. The solution may be switched to be displayed in accordance with each of the plurality of communities. The display column 530 displays the information acquired from the local government system 103.

For example, the solution display section 132 transmits to the local government system 103 an inquiry including the community information, so as to acquire information desired to be displayed together with the solution. The solution display section 132 may acquire the information from the local government system 103 based on the request from the user.

The solution is displayed with reference to the community, so that the user enjoys benefits by using the mother and child health management system 100. Accordingly, a motivation to use the mother and child health management system 100 is increased, leading to an increase of the users as well as each user's constant use of the mother and child health management system 100. Thus, the advice related to everyday life is more highly reliable. The presentation of solutions through the community further enable the construction of a place on the cyber system where the user can share the information with other users who have the same concern, and the user can enjoys the benefits such as grasping more realistic solutions and sharing the concerns. The user may further feel that the user's participation has led to something useful for the other users having the same concern, thereby resulting in the increased motivation to use the mother and child health management system 100.

Particularly, at an early stage of introducing a system for the cohort study, it is difficult for the mother and child health analysis device 112 to provide high-quality advice related to everyday life. In other words, with the mother and child health management system 100 conventionally provided, the user enjoys only limited benefits. On the other hand, the present invention provides the mother and child health management system 100 where the user enjoys the benefits even at the early stage of introducing the system.

(3) Next, in order to remove the access authority, the mother and child health community management device 113 executes the processing as will be described below.

When receiving, from the mobile terminal 101, an evaluation request related to the solution, the mother and child health community management device 113 starts the processing described below. The evaluation request includes the family ID, the community ID, the solution ID, and evaluation information.

The access authority management section 131 of the information management unit 130 refers to the solution DB 170 to search for the record in correspondence to the solution ID. Then, among the records searched, the access authority management section 131 selects the rows in correspondence to the community ID, and based on the evaluation information, updates the evaluation score in the evaluation 403 of the rows selected. For example, when the evaluation information corresponds to a numerical value, the access authority management section 131 adds the numerical value to the evaluation score. When the evaluation information corresponds to a symbol or a character string, the access authority management section 131 converts the evaluation information into a numerical value and adds the numerical value to the evaluation score. Here, with a positive evaluation, the evaluation score is updated to be higher and with a negative evaluation, the evaluation score is updated to be lower.

The access authority management section 131 of the information management unit 130 refers to the local health DB 160 to search for the record of the family including the user of the mobile terminal 101. The access authority management section 131 of the information management unit 130 removes the community ID from the community 304 in the record of the family.

Note that, the access authority management section 131 may inquire the mobile terminal 101 whether or not to leave the community.

When the evaluation for the solution has been completed, the mother and child health community management device 113 determines that the concern shared in the community has been solved, and automatically removes from the community the user that has sent the evaluation. With this configuration, it is possible to use the mother and child health management system 100 with less time and effort.

(4) Next, in order to generate the local information, the mother and child health community management device 113 executes the processing as will be described below.

When receiving, from the local government system 103, the information acquisition request, the mother and child health community management device 113 starts the processing described below. The information acquisition request includes information related to a local area managed by the local government.

The local information generation section 133 of the information management unit 130 refers to the local health DB 160 to search for the record of a family that resides in the local area specified. Specifically, the local information generation section 133 searches for the record where the address corresponding to the local area is stored in the address 302.

The local information generation section 133 analyzes a feature of the community based on the community ID stored in the community 304 of the record searched, so as to generate the local information including the analysis result.

Based on the local information, the local government system 103 displays a screen. FIG. 6 is a diagram showing an example of the screen displayed in the local government system 103 according to the first embodiment.

A screen 600 displays distribution of the users in each of the communities of the local area specified. Additionally, when anyone is allowed to access a local policy service that any other local areas (than the local area specified) provide, the local government system 103 displays the local policy service on the screen 600.

(5) Next, in order to register the solution, the mother and child health community management device 113 executes the processing as will be described below. Here, the solution is registered via the mobile terminal 101 or the mother and child support business system 104.

First, in order to register the solution via the mobile terminal 101, the mother and child health community management device 113 executes the processing below.

The mother and child health community management device 113 receives the solution registration request from the mobile terminal 101. The solution registration request includes the community ID and the solution. Note that, in registration of the solution via the mobile terminal 101, the system is controlled to register only the solution for the community to which the family of the user of the mobile terminal 101 belongs.

The solution registration unit 140 registers each of the solutions in the solution DB 170. Specifically, the solution registration unit 140 adds the corresponding record to the solution DB 170, sets up the identification information in the solution ID 401 of the record added, and sets up the solution in the solution 402. Additionally, the solution registration unit 140 generates a row for the record added, sets up the community ID in a community ID column of the row, and sets up an initial value in an evaluation score column of the row. The initial value is, for example, 0. In this state, the solution registration unit 140 sets up a display flag provided with an expiry date for the record. The display flag is configured to display the solution whether the evaluation score is higher or not. Thus, even when the solution is not yet evaluated, the users are allowed to view the solution. When the expiry date has passed, the solution registration unit 140 removes the display flag from the record.

Next, in order to register the solution via the mother and child support business system 104, the mother and child health community management device 113 executes the processing below.

The mother and child health community management device 113 receives an access from the mother and child support business system 104.

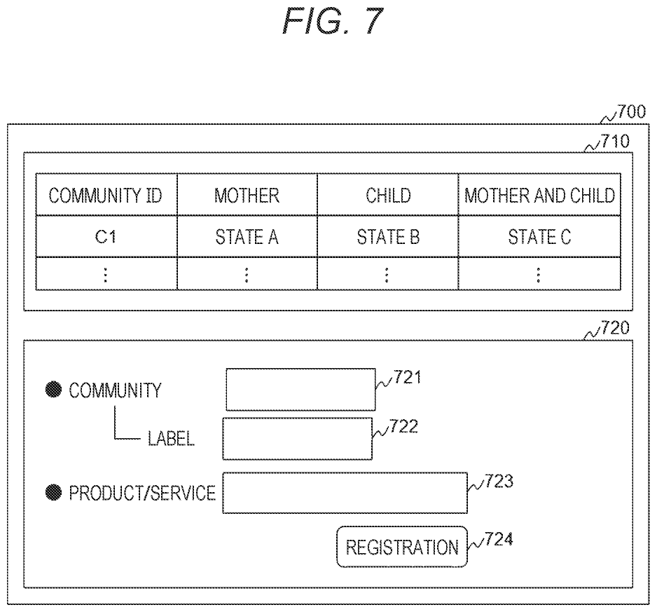

The solution registration unit 140 displays, in the mother and child support business system 104, a registration screen 700 as illustrated in FIG. 7. The registration screen 700 includes a display column 710 and an input column 720. The display column 710 is configured to display the community ID and characteristics of the corresponding community (the state value indicating each of the states). The display column 710 displays, for example, groups of the records stored in the community DB 150. The input column 720 is a column where the information related to the solution is inputted. The input column 720 includes a community input column 721, a label input column 722, a product/service input column 723, and a registration button 724.

The community input column 721 is a column where the community, to which the solution is displayed, is inputted. For example, the community ID is inputted in the community input column 721. The label input column 722 is a column where the label is inputted. The label input column 722 may be blank. The product/service input column 723 is a column where the product or the service provided as the solution is inputted. The registration button 724 is an operation button used to transmit the solution registration request including the values set up in the input column 720. Note that, the input column 720 is merely illustrative and is thus not restrictive. The input column 720 may alternatively be a column where a solution applicable to the plurality of communities is to be inputted.

The user of a mother and child support business refers to the display column 710 to determine the community to which the service or the product is to be provided. The user also sets up a name of the product or the service in the product/service input column 723, sets up the community ID in the community input column 721, and sets up the label in the label input column 722. Then, the user presses the registration button 724.

The mother and child support business system 104 transmits to the mother and child health community management device 113 the solution registration request including the values set up on the registration screen.

The solution registration unit 140 adds the corresponding record to the solution DB 170, and sets up the solution ID and the solution in the record added. Additionally, the solution registration unit 140 adds a row for the record added, and sets up the community ID, the label, and an initial value of the evaluation score for the community in the row. In this state, the solution registration unit 140 sets up the display flag provided with an expiry date for the record.

When the mother and child support business has registered the service or the product, the service or the product may be provided to the user through the mother and child health management system 100. Accordingly, it is possible to further increase the benefits that the user of the mother and child health management system 100 enjoys. Further, it is possible for the mother and child support business to obtain an opportunity to revitalize the business such as advertisement of the services or the products and securing the market.

(6) Next, in order to generate the community, the mother and child health community management device 113 executes the processing as will be described below.

When the community ID indicating the undefined community is set up in the community 304, when the number of the records holding the community ID is greater than a threshold value, or when receiving a generation command, the mother and child health community management device 113 starts the processing described below.

The community generation section 134 of the information management unit 130 refers to the local health DB 160 to search for the record where the community ID indicating the undefined community is set up in the community 304.

The community generation section 134 of the information management unit 130 executes the grouping based on the state values in the state 303 of the record, so as to generate a plurality of the groups. For example, the community generation section 134 executes clustering in the feature space including each of the states, and generates each cluster (group). The clustering method is, for example, a K-means method.

The community generation section 134 of the information management unit 130 selects one of the plurality of groups. The community generation section 134 adds the record in correspondence to the one of the plurality of groups to the community DB 150, and sets up the identification information in the community ID 201 in the corresponding record. Here, the community generation section 134 also calculates an average value of each of the state values for the one of the plurality of groups, and sets up the average value in each of the mother 202, the child 203, and the mother and child 204 in the corresponding record. The community generation section 134 executes the processing above in all of the plurality of groups.

Note that, the community may be generated by use of input data for generating the communities. For example, the community generation section 134 inputs the biological information related to feces of each of a plurality of children to the biological information analysis section 122 of the classification unit 120, and acquires analysis data including the state of the gut microbiome as a state of the corresponding child. In this state, a state of the corresponding mother and a state between the corresponding mother and the corresponding child are described as NULL in the analysis data. The community generation section 134 executes the grouping based on the analysis data to generate the community. The community generation section 134 registers the community information that has been generated in the community DB 150.

Mother and child health management system 100 is configured to automatically generate a community having new characteristics. With this configuration, it is possible to analyze the new community characteristics, accumulate solutions, and display the solutions.

It should be noted that the present invention is not limited to the foregoing embodiment, and various modifications may be included. For example, a detailed description of the foregoing embodiment is to be considered in all respects as merely illustrative for convenience of description, and thus is not restrictive. Further, any addition, removal, and replacement of any other configurations may be partially made to, from, and with the configuration in the foregoing embodiment.

Each of the components, functions, processing units, processing means, or others in the foregoing embodiment may be partially or wholly incorporated into a hardware system, such as an integrated circuit design or a field programmable gate array (FPGA). Further, in the present invention, each of the functions in the foregoing embodiment may be incorporated into a software system including a program code. In this case, a storage medium where the program code is stored is provided to a computer, and a processor included in the computer reads the program code stored in the storage medium. Then, the program code that has been read from the storage medium is used to fulfil the functions of the foregoing embodiment; and in this case, each of the program code and the storage medium storing the program code is provided as an aspect of the present invention. The storage medium for supplying a program code of this type is, for example, a flexible disk, a compact disc read only memory (CD-ROM), a digital versatile disk read only memory (DVD-ROM), a hard disk, the solid state drive (SSD), an optical disk, a magneto-optical disk, a clock-data recovery (CD-R), a magnetic tape, a non-volatile memory card, a ROM, or others.

Additionally, the program code used to fulfil the functions of this embodiment may be, for example, a wide range of programs or script languages such as assembler, C/C++, perl, Shell, hypertext preprocessor (PHP), Python, R, or Java.

Further, the program code of the software system that fulfils the functions of the foregoing embodiment may be distributed via the network, so that the program code is stored in storage means such as the hard disk or the memory of the computer, or the storage medium such as a compact disc rewritable (CD-RW) or the CD-R, and the processor included in the computer reads and executes the program code stored in the storage means or the storage medium.

In the foregoing embodiment, each of a control line and an information line is considered necessary for purpose of description, and thus does not represent all the control lines and the information lines of the product. All the components may be mutually connected.

1. A mother and child health management system provided with at least one computer,
the mother and child health management system connected to a plurality of terminals, through which the mother and child health management system is used, and comprising:
a storage unit configured to store community information to manage a community defined by a combination of a state value indicating a state of a mother, a state value indicating a state of a child, and a state value indicating a state between the mother and the child, and solution information to manage a solution for a concern shared in the community;
a reception unit configured to receive, as analysis information, information inputted as a text from each of the plurality of terminals, the information related to at least any one of the state of the mother and the state of the child, the mother and the child of a family including a user of the each of the plurality of terminals as a member, and biological information from a medical care institution, the biological information related to at least any one of the mother and the child;
a classification unit configured to identify, based on an analysis result of the analysis information, the state value of the mother, the state value of the child, and the state value between the mother and the child, and configured to output each of the state values as state information;
an access authority management section configured to identify a community, to which the family of the user of the terminal belongs, by referring to the community information based on the state information, and configured to provide the terminal with an access authority to receive a display of the solution for the concern shared in the community; and
a solution display section configured to display on the terminal the solution for the concern shared in the community,
wherein
on receipt, from the terminal, of a vote to evaluate the solution for the concern shared in the community to which the family of the user of the terminal belongs, the access authority management section removes from the terminal the access authority to the community to which the family belongs.

2. The mother and child health management system according to claim 1, further comprising a community generation section configured to execute grouping, based on the analysis result of the analysis information received from the each of the plurality of terminals, the grouping of the state values indicating the state of the mother, the state of the child, and the state between the mother and the child, and configured to register a group generated as the community in the community information.

3. The mother and child health management system according to claim 1, further comprising a solution registration unit configured to, on receipt, from a business system to provide the mother and the child with any one of a service and a product, of a request to register any one of the service and the product as the solution for the concern shared in the community, register the any one of the service and the product as the solution in the solution information.

4. The mother and child health management system according to claim 3, wherein on receipt, from the each of the plurality of terminals, of a request to register the solution for the concern shared in the community to which the family of the user of the terminal belongs, the solution registration unit registers the solution in the solution information.

5. The mother and child health management system according to claim 1, wherein
the storage unit stores local health information to manage data related to the community to which the family belongs,
the access authority management section adds, to the data related to the family including the user of the each of the plurality of terminals, information related to the community to which the family belongs, so as to provide the terminal with the access authority to the community, and removes, from the data related to the family including the user of the terminal, the information related to the community to which the family belongs, so as to remove from the terminal the access authority to the community, and
the mother and child health management system further comprises a local information generation section configured to display local information based on the local health information in a local government system, the local information indicating a feature of each of the communities in correspondence to a local area.

6. An information provision method executed by a mother and child health management system provided with at least one computer,
the mother and child health management system connected to a plurality of terminals, through which the mother and child health management system is used, and holding:
community information to manage a community defined by a combination of a state value indicating a state of a mother, a state value indicating a state of a child, and a state value indicating a state between the mother and the child;
solution information to manage a solution for a concern shared in the community; and
local health information to manage data related to a family including a user of each of the plurality of terminals as a member, together with a residential location of the family, a health state of the family, and a community to which the family belongs,
the information provision method comprising the steps performed by at least the one computer of:
receiving, as analysis information, information inputted as a text from the each of the plurality of terminals, the information related to at least any one of the state of the mother and the state of the child, the mother and the child of the family including the user of the terminal, and biological information from a medical care institution, the biological information related to at least any one of the mother and the child;
identifying, based on an analysis result of the analysis information, the state value of the mother, the state value of the child, and the state value between the mother and the child, to output each of the state values as state information;
identifying the community, to which the family of the user of the terminal belongs, by referring to the community information based on the state information, to add information related to the community to the data related to the family, so as to provide the terminal with an access authority to receive a display of the solution for the concern shared in the community;
displaying on the terminal the solution for the concern shared in the community;
displaying local information based on the local health information in a local government system, the local information indicating a feature of each of the communities in correspondence to a local area; and
on receipt, from the terminal, of a vote to evaluate the solution for the concern shared in the community to which the family of the user of the terminal belongs, removing, from the data related to the family, the information related to the community to which the family belongs, so as to remove from the terminal the access authority to the community to which the family belongs.

7. The information provision method according to claim 6, further comprising the step performed by at least the one computer of:
executing grouping, based on the analysis result of the analysis information received from the each of the plurality of terminals, the grouping of the state values indicating the state of the mother, the state of the child, and the state between the mother and the child, to register a group generated as the community in the community information.

8. The information provision method according to claim 6, further comprising the step performed by at least the one computer of:
on receipt, from a business system to provide the mother and the child with any one of a service and a product, of a request to register any one of the service and the product as the solution for the concern shared in the community, registering the any one of the service and the product as the solution in the solution information.

9. The information provision method according to claim 8, further comprising the step performed by at least the one computer of:
on receipt, from the each of the plurality of terminals, of a request to register the solution for the concern shared in the community to which the family of the user of the terminal belongs, registering the solution in the solution information.
